(19) United States
(12) Patent Application Publication
(10) Pub. No.: US 2014/0277011 A1
(43) Pub. Date: Sep. 18, 2014

(54) IMAGING AND TREATMENT DEVICES AND METHODS OF USE THEREOF

(71) Applicant: VOLCANO CORPORATION, San Diego, CA (US)

(72) Inventor: Kristin Meader, San Diego, CA (US)

(73) Assignee: VOLCANO CORPORATION, San Diego, CA (US)

(21) Appl. No.: 14/203,839

(22) Filed: Mar. 11, 2014

Related U.S. Application Data
(60) Provisional application No. 61/777,641, filed on Mar. 12, 2013.

(51) Int. Cl. A61B 17/3207; A61B 19/00

(52) U.S. Cl. CPC A61B 17/320758; A61B 19/5244
USPC 606/159

(57) ABSTRACT

The invention generally relates to imaging and treatment devices and methods of use thereof. In certain aspects, the invention provides a steerable imaging and treatment device. The device includes an elongate steerable body. An imaging apparatus is at least partially housed in the body and configured to image in a forward direction. A rotatable head is coupled to a distal end of the body, and includes an abrasive surface.

### RELATED APPLICATION

The present application claims the benefit of and priority to U.S. provisional patent application Ser. No. 61/777,641, filed Mar. 12, 2013, the content of which is incorporated by reference herein in its entirety.

### FIELD OF THE INVENTION

The invention generally relates to imaging and treatment devices and methods of use thereof.

### BACKGROUND

Cardiovascular disease frequently arises from the accumulation of atheromatous deposits on inner walls of vascular lumen, particularly the arterial lumen of the coronary and other vasculature, resulting in a condition known as atherosclerosis. These deposits can have widely varying properties, with some deposits being relatively soft and others being fibrous and/or calcified. In the latter case, the deposits are frequently referred to as plaque. For example, an artery may be completely blocked with plaque, in what is referred to as a chronic total occlusion. Chronic total occlusions are responsible for clinically significant decreases in blood flow. In addition, chronic total occlusions often mean more significant intervention, such as coronary artery bypass surgery.

Interventional vascular procedures such as atherectomy, are used to eliminate chronic total occlusions from the vasculature system. During an atherectomy procedure, an atherectomy catheter is inserted into a blood vessel and passed to the site of the obstruction. Contrast material is injected through the catheter to visualize the obstruction using an external x-ray imaging system. The catheter typically includes a radiopaque marker so that it also can be visualized by the external imaging system while the catheter is in the vessel. The catheter's removal assembly, such as a rotating abrasive head engages with the obstruction to allow removal of the atheromatous material on the inner wall of the vessel. The treatment area is visualized by the external x-ray imaging system during and subsequent to the removal to ensure that the obstruction has been removed by the catheter. If atheromatous material remains, the process is repeated until the obstruction is removed.

It is known to include a forward looking imaging sensor with an atherectomy catheter (see U.S. Pat. No. 5,100,424).

### SUMMARY

The invention recognizes that a problem with known atherectomy catheters is that they are not steerable. As such, atherectomy catheters create paths within chronic total occlusions that take the catheter out of the true vessel lumen and into the subintimal space. The invention generally relates to steerable devices that allow for steering of the device based on real-time image data so that the device remains within the vessel while advancing the device through a chronic total occlusion. Aspects of the invention are accomplished by providing a steerable device with an integrated forward looking imaging assembly that is coupled to a body of the device. This increases safety and allows an operator to better direct the atherectomy.

In certain aspects, the invention provides a steerable imaging and treatment device. The device includes an elongate steerable body. An imaging apparatus is at least partially housed in the body and configured to image in a forward direction. A rotatable head is coupled to a distal end of the body, and includes an abrasive surface. The abrasive surface may include abrasive material bonded to the head. Exemplary abrasive material includes diamond powder, fused silica, tungsten carbide, aluminum oxide, or boron carbide. In certain embodiments, the abrasive surface is the result of roughening of the rotatable head to cause grooves, crevasses, indentations, etc., in the surface of the head. In certain embodiments, the rotatable head is roughened and bonded with abrasive material. Devices of the present invention may be used in a variety of body lumens, including but not limited to intravascular lumens such as coronary arteries. Typically, devices of the invention are used to remove occlusive material, such as atherosclerotic plaque, from vascular lumens, but they may alternatively or also be used to remove one or more other materials.

In devices and methods of the invention, an imaging assembly is coupled to the body and positioned to image the opening in the device. Any imaging assembly may be used with devices and methods of the invention, such as opto-acoustic sensor apparatuses, intravascular ultrasound (IVUS) or optical coherence tomography (OCT). In certain embodiments, the imaging assembly includes at least one opto-acoustic sensor. Generally, the opto-acoustic sensor will include an optical fiber having a blazed fiber Bragg grating, a light source that transmits light through the optical fiber, and a photoacoustic transducer material positioned so that it receives light diffracted by the blazed fiber Bragg grating and emits ultrasonic imaging energy. The sensor may be positioned on an internal wall of the device, opposite the opening. In certain embodiments, the at least one sensor is a plurality of sensors and the sensors are arranged in a semi-circle.

In another aspect, the invention provides methods for removing an occlusion from a vessel. Methods of the invention involve providing a steerable imaging and treatment device. The device includes an elongate steerable body. An imaging apparatus is at least partially housed in the body and configured to image in a forward direction. A rotatable head is coupled to a distal end of the body, and includes an abrasive surface. Methods of the invention additionally involve inserting the device into a vessel. The rotating head is contacted to an occlusion in the vessel while imaging the occlusion. The device is steered based on real-time image data to remain within the vessel while advancing the device through the occlusion.

### BRIEF DESCRIPTION OF THE DRAWINGS

FIG. 1 is an illustration of an imaging and treating device of the invention.

FIG. 2-3 are illustrations of a flexing steering mode.

FIG. 4 is a schematic diagram of a conventional optical fiber.

FIG. 5 is a cross-sectional schematic diagram illustrating generally one example of a distal portion of an imaging assembly that combines an acousto-optic Fiber Bragg Grating (FBG) sensor with an photoacoustic transducer.

FIG. 6 is a schematic diagram of a Fiber Bragg Grating based sensor

FIG. 7 is a cross-sectional schematic diagram illustrating generally one example of the operation of a blazed grating FBG photoacoustic transducer.

FIG. 8 is a schematic diagram illustrating generally one technique of generating an image by rotating the blazed FBG optical-to-acoustic and acoustic-to-optical combined transducer and displaying the resultant series of radial image lines to create a radial image.

FIG. 9 is a schematic diagram that illustrates generally one such phased array example, in which the signal to/from each array transducer is combined with the signals from the other transducers to synthesize a radial image line.

FIG. 10 is a schematic diagram that illustrates generally an example of a side view of a distal portion of a device.

FIG. 11 is a schematic diagram that illustrates generally one example of a cross-sectional side view of a distal portion of a device.

FIG. 12 is a block diagram illustrating generally one example of the imaging assembly and associated interface components.

FIG. 13 is a block diagram illustrating generally another example of the imaging assembly and associated interface components, including tissue characterization and image enhancement modules.

### DETAILED DESCRIPTION

The invention generally relates to imaging and treatment devices and methods of use thereof. In certain aspects, the invention provides a steerable imaging and treatment device. The device includes an elongate steerable body. An imaging apparatus is at least partially housed in the body and configured to image in a forward direction. A rotatable head is coupled to a distal end of the body, and includes an abrasive surface.

In certain embodiments, the device is a catheter and the body is a catheter body. The catheter and catheter body are configured for intraluminal introduction to the target body lumen. The dimensions and other physical characteristics of the catheter bodies will vary significantly depending on the body lumen that is to be accessed. In the exemplary case of atherectomy catheters intended for intravascular introduction, the proximal portions of the catheter bodies will typically be very flexible and suitable for introduction over a guidewire to a target site within the vasculature. In particular, catheters can be intended for “over-the-wire” introduction when a guidewire channel extends fully through the catheter body or for “rapid exchange” introduction where the guidewire channel extends only through a distal portion of the catheter body. In other cases, it may be possible to provide a fixed or integral coil tip or guidewire tip on the distal portion of the catheter or even dispense with the guidewire entirely. For convenience of illustration, guidewires will not be shown in all embodiments, but it should be appreciated that they can be incorporated into any of these embodiments.

Catheter bodies intended for intravascular introduction will typically have a length in the range from 50 cm to 200 cm and an outer diameter in the range from 1 French to 12 French (0.33 mm: 1 French), usually from 3 French to 9 French. In the case of coronary catheters, the length is typically in the range from 125 cm to 200 cm, the diameter is preferably below 8 French, more preferably below 7 French, and most preferably in the range from 2 French to 7 French. Catheter bodies will typically be composed of an organic polymer that is fabricated by conventional extrusion techniques. Suitable polymers include polyvinylchloride, polyurethanes, polyesters, polytetrafluoroethylenes (PTFE), silicone rubbers, natural rubbers, and the like. Optionally, the catheter body may be reinforced with braid, helical wires, coils, axial filaments, or the like, in order to increase rotational strength, column strength, toughness, pushability, and the like. Suitable catheter bodies may be formed by extrusion, with one or more channels being provided when desired. The catheter diameter can be modified by heat expansion and shrinkage using conventional techniques. The resulting catheters will thus be suitable for introduction to the vascular system, often the coronary arteries, by conventional techniques.

The distal portion of the catheters of the present invention may have a wide variety of forms and structures. In many embodiments, a distal portion of the catheter is more rigid than a proximal portion, but in other embodiments the distal portion may be equally as flexible as the proximal portion. One aspect of the present invention provides catheters having a distal portion with a reduced rigid length. The reduced rigid length can allow the catheters to access and treat tortuous vessels and small diameter body lumens. In most embodiments a rigid distal portion or housing of the catheter body will have a diameter that generally matches the proximal portion of the catheter body, however, in other embodiments, the distal portion may be larger or smaller than the flexible portion of the catheter.

A rigid distal portion of a catheter body can be formed from materials that are rigid or which have very low flexibilities, such as metals, hard plastics, composite materials, NiTi, steel with a coating such as titanium nitride, tantalum, ME-92 (antibacterial coating material), diamonds, or the like. Most usually, the distal end of the catheter body will be formed from stainless steel or platinum/iridium. The length of the rigid distal portion may vary widely, typically being in the range from 5 mm to 35 mm, more usually from 10 mm to 25 mm, and preferably between 6 mm and 8 mm. In contrast, conventional catheters typically have rigid lengths of approximately 16 mm.

### Steerability

FIG. 1 illustratively depicts an embodiment of the catheter assembly 10 including a catheter body/shaft 12. The catheter shaft 12 is a generally flexible elongate member having a distal segment 14, a proximal segment 16, and at least one lumen (not shown). The proximal segment 16 is attached to a handle 18. The handle 18 includes, by way of example, a housing 20, a steering actuator 24.

The actuator 24 is manipulated by a user moving an exposed control surface of the actuator 24 (using a finger/thumb) lengthwise along the length of the housing 20 of the handle 18 (as opposed to across the width of the handle 18). In alternative embodiments, thumb-controlled slider actuators replace the rotating knobs. The distal segment 14 is, by way of example, 10 cm long. However, an exemplary range for the length of the distal segment 14 is from 5 cm to 20 cm. A tip of the distal segment 14 has a generally smaller diameter than the diameter of the proximal segment 16 of the catheter shaft. The catheter shaft 12 is made, by way of example, of engineered nylon (polyether block amide) and includes a tube or tubing, alternatively called a catheter tube or catheter tubing that has at least one lumen.

In the illustrative example in FIG. 1, the steering actuator 24 is accessible (have exposed control surfaces through the housing 20) on two sides of the handle 18. A strain relief 26 protects the catheter shaft 12 at a point where the catheter shaft proximal segment 16 meets the handle 18. A cable 28 connects the handle 18 to a connector 30. The connector 30, which can be any of many possible configurations, is configured to interconnect with an imaging system for processing, storing, manipulating, and displaying data obtained from signals generated by a sensor mounted at the distal segment 14 of the catheter shaft 12.

FIGS. 2 and 3 illustrate distal segment 14 flexing steering affected by the actuator 24 in the first embodiment. The catheter 12 is flexed, using the actuator 24, from a straight configuration as illustrated in FIG. 2 into a flexed steering configuration, as illustrated in FIG. 3. In addition, the catheter 12's distal segment 14 is steerable into any number of flexed positions in between the straight configuration of FIG. 2 and the flexed configuration of FIG. 3, and can even be flexed beyond the configuration of FIG. 3. The catheter is capable of flexing past the 90°point in each direction and has an angular range of 0° to 150° from the straight or neutral configuration. The second direction is similar to what has been illustrated in FIG. 3, and it can be appreciated that it is simply the mirror image of the configuration of FIG. 3 illustrated for the first direction.

To affect flexing the distal segment 14 in the manner described above, the second steering actuator 24 (e.g., knob) is turned in a first rotational direction with respect to the relatively fixed position handle 18. Rotating the actuator 24 in the first direction causes a first steering wire to apply tension to a steering bulkhead 38 forcing the distal segment 14 of the catheter shaft 12 to bend at bending joint 15 (see, FIG. 3). In order to flex the catheter in the opposite direction, the second steering actuator 24 is turned in an opposing second rotational direction with respect to the handle 18. This causes a second steering wire to apply tension to an opposite side of steering bulkhead 38, forcing the catheter to bend in an opposite direction at the bending joint 15. The catheter assembly 10, by way of example, supports bidirectional flexed steering by at least 150 degrees in each direction from a neutral or straight catheter position. Using the combination of these two steering modes (rotational and flexing) is much more intuitive to the user than a steering mechanism based solely on either rotation or flexing—but not both. In an example of a method for using the catheter assembly 10 having both rotational and flex steering, the rotating abrasive head 34 is first placed into a desired location of the body. While visualizing the rotating abrasive head 34, such as with ultrasound, the second steering actuator 24 is adjusted until the catheter orientation is close to the desired orientation.

Cable wires from the connector extend through a proximal orifice. The catheter steering mechanisms and signal wire bundle extend through distal orifice. The lower portion of the thumb and the two smallest fingers comfortably grip the handle at a grip area. The shape of the handle and positioning of the actuators permits easy access for the thumb on the top of the handle and either the index or middle finger on the bottom of the handle to manipulate the steering actuator 24 while maintaining hold on the grip area of the handle.

In certain embodiments, a lock lever protrude slightly above the outer edges/diameters of the steering actuator 24. While in the resting locked position shown, the locking mechanisms controlled by the levers do not allow 24 to be moved, thus maintaining the catheter 10 in its desired flex state. While a user's thumb manipulates one of the actuator 24, the associated one of the lock lever is held down slightly by the thumb, releasing the corresponding locking mechanism and allowing the actuator to be moved (e.g., the knob rotates). After the actuator 24 is moved to the desired position and the thumb is taken off the lock lever, the corresponding lock automatically engages the actuator 24, holding the actuator 24 in the desired position until the next time it is to be moved.

### Imaging Apparatus

Devices of the invention also include an imaging assembly 32 coupled to the body 12. The imaging assembly may be placed distal or proximal to the abrasion head 34, or positioned elsewhere. The imaging assembly 32 is configured to image in a forward direction.

Any imaging assembly may be used with devices and methods of the invention, such as optical-acoustic imaging apparatus, intravascular ultrasound (IVUS), forward-looking intravascular ultrasound (FLIVUS) or optical coherence tomography (OCT). In certain embodiments, the imaging assembly is an optical-acoustic imaging apparatus. Exemplary optical-acoustic imaging sensors are shown for example in, U.S. Pat. No. 7,245,789; U.S. Pat. Nos. 7,447,388; 7,660,492; U.S. Pat. No. 8,059,923; US 2012/0108943; and US 2010/0087732, the content of each of which is incorporated by reference herein in its entirety. Additional optical-acoustic sensors are shown for example in U.S. Pat. No. 6,659,957; U.S. Pat. No. 7,527,594; and US 2008/0119739, the content of each of which is incorporated by reference herein in its entirety.

An exemplary optical-acoustic imaging apparatus includes a photoacoustic transducer and a blazed Fiber Bragg grating. Optical energy of a specific wavelength travels down a fiber core of optical fiber and is reflected out of the optical fiber by the blazed grating. The outwardly reflected optical energy impinges on the photoacoustic material. The photoacoustic material then generates a responsive acoustic impulse that radiates away from the photoacoustic material toward nearby biological or other material to be imaged. Acoustic energy of a specific frequency is generated by optically irradiating the photoacoustic material at a pulse rate equal to the desired acoustic frequency.

The optical-acoustic imaging apparatus utilizes at least one and generally more than one optical fiber, for example but not limited to a glass fiber at least partly composed of silicon dioxide. The basic structure of a generic optical fiber is illustrated in FIG. 4, which fiber generally consists of layered glass cylinders. There is a central cylinder called the core 1. Surrounding this is a cylindrical shell of glass, possibly multilayered, called the cladding 2. This cylinder is surrounded by some form of protective jacket 3, usually of plastic (such as acrylate). For protection from the environment and more mechanical strength than jackets alone provide, fibers are commonly incorporated into cables. Typical cables have a polyethylene sheath 4 that encases the fibers within a strength member 5 such as steel or Kevlar strands.

FIG. 5 is a cross-sectional schematic diagram illustrating generally one example of a distal portion of an imaging assembly that combines an acousto-optic Fiber Bragg Grating (FBG) sensor 100 with an photoacoustic transducer 325. The optical fiber includes a blazed Fiber Bragg grating. Fiber Bragg Gratings form an integral part of the optical fiber structure and can be written intracore during manufacture or after manufacture. As illustrated in FIG. 6, when illuminated by a broadband light laser 7, a uniform pitch Fiber Bragg Grating element 8 will reflect back a narrowband component centered about the Bragg wavelength λ given by λ=2nλ, where n is the index of the core of the fiber and λ represents the grating period. Using a tunable laser 7 and different grating periods (each period is approximately 0.5 μm) situated in different positions on the fiber, it is possible to make independent measurement in each of the grating positions.

Referring back to FIG. 5, unlike an unblazed Bragg grating, which typically includes impressed index changes that are substantially perpendicular to the longitudinal axis of the fiber core 115 of the optical fiber 105, the blazed Bragg grating 330 includes obliquely impressed index changes that are at a nonperpendicular angle to the longitudinal axis of the optical fiber 105. As mentioned above, a standard unblazed FBG partially or substantially fully reflects optical energy of a specific wavelength traveling down the axis of the fiber core 115 of optical fiber 105 back up the same axis. Blazed FBG 330 reflects this optical energy away from the longitudinal axis of the optical fiber 105. For a particular combination of blaze angle and optical wavelength, the optical energy will leave blazed FBG 330 substantially normal (i.e., perpendicular) to the longitudinal axis of the optical fiber 105. In the illustrative example of FIG. 22, an optically absorptive photoacoustic material 335 (also referred to as a “photoacoustic” material) is placed on the surface of optical fiber 105. The optically absorptive photoacoustic material 335 is positioned, with respect to the blazed grating 330, so as to receive the optical energy leaving the blazed grating. The received optical energy is converted in the optically absorptive material 335 to heat that expands the optically absorptive photoacoustic material 335. The optically absorptive photoacoustic material 335 is selected to expand and contract quickly enough to create and transmit an ultrasound or other acoustic wave that is used for acoustic imaging of the region of interest.

FIG. 7 is a cross-sectional schematic diagram illustrating generally one example of the operation of photoacoustic transducer 325 using a blazed Bragg grating 330. Optical energy of a specific wavelength, λ₁, travels down the fiber core 115 of optical fiber 105 and is reflected out of the optical fiber 105 by blazed grating 330. The outwardly reflected optical energy impinges on the photoacoustic material 335. The photoacoustic material 335 then generates a responsive acoustic impulse that radiates away from the photoacoustic material 335 toward nearby biological or other material to be imaged. Acoustic energy of a specific frequency is generated by optically irradiating the photoacoustic material 335 at a pulse rate equal to the desired acoustic frequency.

In another example, the photoacoustic material 335 has a thickness 340 (in the direction in which optical energy is received from blazed Bragg grating 330) that is selected to increase the efficiency of emission of acoustic energy. In one example, thickness 340 is selected to be about ¼ the acoustic wavelength of the material at the desired acoustic transmission/reception frequency. This improves the generation of acoustic energy by the photoacoustic material.

In yet a further example, the photoacoustic material is of a thickness 300 that is about ¼ the acoustic wavelength of the material at the desired acoustic transmission/reception frequency, and the corresponding glass-based optical fiber sensing region resonant thickness 300 is about ½ the acoustic wavelength of that material at the desired acoustic transmission/reception frequency. This further improves the generation of acoustic energy by the photoacoustic material and reception of the acoustic energy by the optical fiber sensing region.

In one example of operation, light reflected from the blazed grating excites the photoacoustic material in such a way that the optical energy is efficiently converted to substantially the same acoustic frequency for which the FBG sensor is designed. The blazed FBG and photoacoustic material, in conjunction with the aforementioned FBG sensor, provide both a transmit transducer and a receive sensor, which are harmonized to create an efficient unified optical-to-acoustic-to-optical transmit/receive device. In one example, the optical wavelength for sensing is different from that used for transmission. In a further example, the optical transmit/receive frequencies are sufficiently different that the reception is not adversely affected by the transmission, and vice-versa.

FIG. 8 is a schematic diagram illustrating generally one technique of generating an image of biological material and a vessel wall 600 through an opening in a device. The technique involves rotating the blazed FBG optical-to-acoustic and acoustic-to-optical combined transducer 500 and displaying the resultant series of radial image lines to create a radial image. In another example, phased array images are created using a substantially stationary (i.e., non-rotating) set of multiple FBG sensors, such as FBG sensors 500A-J. FIG. 9 is a schematic diagram that illustrates generally one such phased array example, in which the signal to/from each array transducer 500A-J is combined with the signals from one or more other transducers 500A-J to synthesize a radial image line. In this example, other image lines are similarly synthesized from the array signals, such as by using specific changes in the signal processing used to combine these signals.

FIG. 10 is a schematic diagram that illustrates generally an example of a side view of a distal portion 800 of an elongate device 805. In this example, the distal portion 800 of the device 805 includes one or more openings 810A, 810B, . . . , 810N located slightly or considerably proximal to a distal tip 815 of the device 805. Each opening 810 includes one or more optical-to-acoustic transducers 325 and a corresponding one or more separate or integrated acoustic-to-optical FBG sensors 100. In one example, each opening 810 includes an array of blazed FBG optical-to-acoustic and acoustic-to-optical combined transducers 500 (such as illustrated in FIG. 10) located slightly proximal to distal tip 815 of device 805 having mechanical properties that allow the device 805 to be guided through a vascular or other lumen.

FIG. 11 is a schematic diagram that illustrates generally one example of a cross-sectional side view of a distal portion 900 of another device 905. In this example, optical fibers 925 are distributed around a bottom portion of device 905. In this example, the optical fibers 925 are at least partially embedded in a polymer matrix or other binder material that bonds the optical fibers 925 to the device 905. The binder material may also contribute to the torsion response of the resulting device 905. In one example, the optical fibers 925 and binder material is overcoated with a polymer or other coating 930, such as for providing abrasion resistance, optical fiber protection, and/or friction control.

In one example, before the acoustic transducer(s) is fabricated, the device 905 is assembled, such as by binding the optical fibers 925 to the device 905, and optionally coating the device 905. The opto-acoustic transducer(s) are then integrated into the imaging assembly, such as by grinding one or more grooves in the device wall at locations of the opto-acoustic transducer window 810. In a further example, the depth of these groove(s) in the optical fiber(s) 925 defines the resonant structure(s) of the opto-acoustic transducer(s).

After the opto-acoustic transducer windows 810 have been defined, the FBGs added to one or more portions of the optical fiber 925 within such windows 810. In one example, the FBGs are created using an optical process in which the portion of the optical fiber 925 is exposed to a carefully controlled pattern of UV radiation that defines the Bragg gratings. Then, a photoacoustic material is deposited or otherwise added in the transducer windows 810 over respective Bragg gratings. One example of a suitable photoacoustic material is pigmented polydimethylsiloxane (PDMS), such as a mixture of PDMS, carbon black, and toluene.

FIG. 12 is a block diagram illustrating generally one example of the imaging assembly 905 and associated interface components. The block diagram of FIG. 12 includes the imaging assembly 905 that is coupled by optical coupler 1305 to an optoelectronics module 1400. The optoelectronics module 1400 is coupled to an image processing module 1405 and a user interface 1410 that includes a display providing a viewable still and/or video image of the imaging region near one or more acoustic-to-optical transducers using the acoustically-modulated optical signal received therefrom. In one example, the system 1415 illustrated in the block diagram of FIG. 12 uses an image processing module 1405 and a user interface 1410 that are substantially similar to existing acoustic imaging systems.

FIG. 13 is a block diagram illustrating generally another example of the imaging assembly 905 and associated interface components. In this example, the associated interface components include a tissue characterization module 1420 and an image enhancement module 1425. In this example, an input of tissue characterization module 1420 is coupled to an output from optoelectronics module 1400. An output of tissue characterization module 1420 is coupled to at least one of user interface 1410 or an input of image enhancement module 1425. An output of image enhancement module 1425 is coupled to user interface 1410, such as through image processing module 1405.

In this example, tissue characterization module 1420 processes a signal output from optoelectronics module 1400. In one example, such signal processing assists in distinguishing blood clots from nearby vascular tissue. Such clots can be conceptualized as including, among other things, cholesterol, thrombus, and loose connective tissue that build up within a blood vessel wall. Calcified plaque typically reflects ultrasound better than the nearby vascular tissue, which results in high amplitude echoes. Soft plaques, on the other hand, produce weaker and more texturally homogeneous echoes. These and other differences distinguishing between plaque deposits and nearby vascular tissue are detected using tissue characterization signal processing techniques.

For example, such tissue characterization signal processing may include performing a spectral analysis that examines the energy of the returned ultrasound signal at various frequencies. A blood clot deposit will typically have a different spectral signature than nearby vascular tissue without such clot, allowing discrimination therebetween. Such signal processing may additionally or alternatively include statistical processing (e.g., averaging, filtering, or the like) of the returned ultrasound signal in the time domain. Other signal processing techniques known in the art of tissue characterization may also be applied. In one example, the spatial distribution of the processed returned ultrasound signal is provided to image enhancement module 1425, which provides resulting image enhancement information to image processing module 1405. In this manner, image enhancement module 1425 provides information to user interface 1410 that results in a displaying blood clots in a visually different manner (e.g., by assigning clots a discernable color on the image) than other portions of the image. Other image enhancement techniques known in the art of imaging may also be applied.

The opto-electronics module 1400 may include one or more lasers and fiber optic elements. In one example, such as where different transmit and receive wavelengths are used, a first laser is used for providing light to the imaging assembly 905 for the transmitted ultrasound, and a separate second laser is used for providing light to the imaging assembly 905 for being modulated by the received ultrasound. In this example, a fiber optic multiplexer couples each channel (associated with a particular one of the optical fibers 925) to the transmit and receive lasers and associated optics. This reduces system complexity and costs.

In one example, the sharing of transmit and receive components by multiple guidewire channels is possible at least in part because the acoustic image is acquired over a relatively short distance (e.g., millimeters). The speed of ultrasound in a human or animal body is slow enough to allow for a large number of transmit/receive cycles to be performed during the time period of one image frame. For example, at an image depth (range) of about 2 cm, it will take ultrasonic energy approximately 26 microseconds to travel from the sensor to the range limit, and back. In one such example, therefore, an about 30 microseconds transmit/receive (T/R) cycle is used. In the approximately 30 milliseconds allotted to a single image frame, up to 1,000 T/R cycles can be carried out. In one example, such a large number of T/R cycles per frame allows the system to operate as a phased array even though each sensor is accessed in sequence. Such sequential access of the photoacoustic sensors in the guidewire permits (but does not require) the use of one set of T/R opto-electronics in conjunction with a sequentially operated optical multiplexer. In one example, instead of presenting one 2-D slice of the anatomy, the system is operated to provide a 3-D visual image that permits the viewing of a desired volume of the patient's anatomy or other imaging region of interest. This allows the physician to quickly see the detailed spatial arrangement of structures, such as lesions, with respect to other anatomy.

In one example, in which the imaging assembly 905 includes 30 sequentially-accessed optical fibers having up to 10 photoacoustic transducer windows per optical fiber, 30×10=300 T/R cycles are used to collect the image information from all the openings for one image frame. This is well within the allotted 1,000 such cycles for a range of 2 cm, as discussed above. Thus, such an embodiment allows substantially simultaneous images to be obtained from all 10 openings at of each optical fiber at video rates (e.g., at about 30 frames per second for each transducer window). This allows real-time volumetric data acquisition, which offers a distinct advantage over other imaging techniques. Among other things, such real-time volumetric data acquisition allows real-time 3-D vascular imaging, including visualization of the topology of a blood vessel wall, the extent and precise location of blood clots, and, therefore, the ability to identify blood clots.

In another embodiment, the imaging assembly uses intravascular ultrasound (IVUS). IVUS imaging assemblies and processing of IVUS data are described for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, and other references well known in the art relating to intraluminal ultrasound devices and modalities.

In another embodiment, the imaging assembly uses optical coherence tomography (OCT). OCT is a medical imaging methodology using a miniaturized near infrared light-emitting probe. As an optical signal acquisition and processing method, it captures micrometer-resolution, three-dimensional images from within optical scattering media (e.g., biological tissue). Recently it has also begun to be used in interventional cardiology to help diagnose coronary artery disease. OCT allows the application of interferometric technology to see from inside, for example, blood vessels, visualizing the endothelium (inner wall) of blood vessels in living individuals.

OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety.

### Abrasion Head

In the present invention, an abrasive head is provided at the distal end of the catheter body. The abrasive head may be comprised of a unitary abrasive material or may be a composite of a support material and an abrasive surface material affixed to the support. When an abrasive surface material is employed it may be adhesively bonded to the support by methods well known to those skilled in the art. Preferred abrasive materials comprising the surface of the abrasive head include fused silica, diamond powder, tungsten carbide, aluminum oxide, boron carbide, and other ceramic materials. Suitable abrasive materials are available commercially from Norton Company, Worcester, Mass.

When a composite abrasive surface and solid support is employed as the abrasive head of the present invention, a suitable support material must be identified. Generally, a suitable support will be any solid material that bonds effectively to the abrasive surface material with an adhesive. The support further must withstand the inertial and impact forces experienced during operation of the abrasive head. A preferred support material is stainless steel. Also, the support preferably has an axial lumen to allow passage of a guidewire therethrough.

Typically, the abrasive head is attached to a drive means via a coupling provided at its proximal end. The drive means will extend axially through a lumen provided in the catheter body to a point external the patient's body. The drive means is connected to a drive motor which provides the power for driving the abrasive head. The drive means preferably includes an internal lumen which permits passage of a guidewire if desired.

Operation of the drive means is such that one revolution of the drive means causes one revolution of the abrasive head. During operation, the drive means will usually make 200-100,000 revolutions per minute. Also, translation of the drive means relative to the catheter body causes a translation of the abrasive head relative to the catheter body. Translation and rotation of the abrasive head via the drive means is afforded so as to permit the head to contact stenotic material that penetrates the aperture of the housing. The construction and operation of drive means suitable for use in the present invention is further described in U.S. Pat. No. 4,794,931, which is incorporated by reference herein.

Grooves (or channels) are optionally provided axially along the exterior wall of the abrasive head to assist passage of dislodged material from the distal-most end of the device. For example, the rotatable abrasive head may include two grooves. An abrasive head having grooves may have only one groove or may have a plurality of grooves. Preferably, the grooves will be spaced equally about the periphery of the head; however, such equal spacing is not necessary.

### Methods of Use

Some exemplary methods of the present invention will now be described. One method of the present invention includes delivering a device to a target site in the body lumen. Once at or near the target site, the imaging assembly is activated. This allows the images of the tissue and occlusions ahead of the device to be obtained and transmitted back to an operator prior to starting a procedure.

The device can be percutaneously advanced through a guide catheter or sheath and over a conventional or imaging guidewire using conventional interventional techniques. The device can be advanced over the guidewire and out of the guide catheter to the diseased area. If there is a cover, the opening will typically be closed. Although, a cover is not required. The device will typically have at least one hinge or pivot connection to allow pivoting about one or more axes of rotation to enhance the delivery of the catheter into the tortuous anatomy without dislodging the guide catheter or other sheath. The device can be positioned proximal of the blood clot.

Once positioned, biological material may be removed from the body lumen by activating the abrasive head. Thereafter, the operator can steer the device through the occlusion using real-time image data provided by the imaging apparatus. The rotating abrasion head breaks-up the occlusion. When it is determined that the blood clot or other obstructive material has been removed, the catheter can be removed from the body lumen.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A steerable imaging and treatment device, the device comprising:
an elongate steerable body;
an imaging apparatus at least partially housed in the body and configured to image in a forward direction; and
a rotatable head coupled to a distal end of the body, the head comprising an abrasive surface.

2. The device according to claim 1, wherein the abrasive surface comprises abrasive material bonded to the head.

3. The device according to claim 2, wherein the abrasive material is comprised of a material selected from the group consisting of diamond powder, fused silica, tungsten carbide, aluminum oxide, and boron carbide.

4. The device according to claim 1, wherein the imaging apparatus is selected from the group consisting of intravascular ultrasound and optical coherence tomography.

5. The device according to claim 1, wherein the imaging apparatus comprises an optical fiber.

6. The device according to claim 5, wherein the optical fiber comprises a fiber Bragg grating.

7. The device according to claim 6, wherein the fiber Bragg grating is a blazed fiber Bragg grating.

8. The device according to claim 1, wherein the imaging apparatus is at a distal region of the elongated body.

9. A method for removing an occlusion from a vessel, the method comprising:
providing a device comprising an elongate steerable body; an imaging apparatus at least partially housed in the body and configured to image in a forward direction; and a rotatable head coupled to a distal end of the body, the head comprising an abrasive surface;
inserting the device into a vessel;
contacting the rotating head to an occlusion in the vessel while imaging the occlusion; and
steering the device based on real-time image data to remain within the vessel while advancing the device through the occlusion, thereby removing the occlusion from the vessel.

10. The device according to claim 9, wherein the abrasive surface comprises abrasive material bonded to the head.

11. The method according to claim 10, wherein the abrasive material is comprised of a material selected from the group consisting of diamond powder, fused silica, tungsten carbide, aluminum oxide, and boron carbide.

12. The method according to claim 9, wherein the imaging apparatus is selected from the group consisting of intravascular ultrasound and optical coherence tomography.

13. The method according to claim 9, wherein the imaging apparatus comprises an optical fiber.

14. The method according to claim 13, wherein the optical fiber comprises a fiber Bragg grating.

15. The method according to claim 14, wherein the fiber Bragg grating is a blazed fiber Bragg grating.

16. The method according to claim 9, wherein the imaging apparatus is at a distal region of the elongated body.
